(19) United States
(12) Patent Application Publication
(10) Pub. No.: US 2020/0383662 A1
(43) Pub. Date: Dec. 10, 2020

(54) ULTRASONIC DIAGNOSTIC APPARATUS, CONTROL METHOD FOR ULTRASONIC DIAGNOSTIC APPARATUS, AND CONTROL PROGRAM FOR ULTRASONIC DIAGNOSTIC APPARATUS

(71) Applicant: Konica Minolta, Inc., Tokyo (JP)

(72) Inventors: Makiko URABE, Yokohama-shi (JP); Yoshihiro TAKEDA, Tokyo (JP); Kazuya TAKAGI, Tokyo (JP); Akihiro KAWABATA, Tokyo (JP)

(21) Appl. No.: 16/887,805

(22) Filed: May 29, 2020

(30) Foreign Application Priority Data
Jun. 7, 2019 (JP) 2019-107117

(51) Int. Cl. A61B 8/08; A61B 8/14; A61B 8/06; A61B 8/00

(52) U.S. Cl. CPC A61B 8/5246; A61B 8/14; A61B 8/488; A61B 8/463; A61B 8/06

(57) ABSTRACT

An ultrasonic diagnostic apparatus includes: a tomographic image generator that generates a tomographic image showing an inside of a subject, based on a received signal relating to an ultrasonic echo of a first ultrasonic beam transmitted to the subject; a Doppler processor that detects a Doppler shift in frequency from a transmission frequency of a second ultrasonic beam, based on a received signal relating to an ultrasonic echo of the second ultrasonic beam transmitted to the subject; a hardware processor that detects a blood vessel position in the subject based on image information about the tomographic image and sets an angle correction value relating to an intersection angle between a blood vessel extending direction at the blood vessel position and a beam direction of the second ultrasonic beam; and a display processor that generates a Doppler spectrum image representing a distribution of blood flow rate at the blood vessel position.

The entire disclosure of Japanese patent Application No. 2019-107117, filed on Jun. 7, 2019, is incorporated herein by reference in its entirety.

### BACKGROUND

### Technological Field

The present disclosure relates to an ultrasonic diagnostic apparatus, a control method for an ultrasonic diagnostic apparatus, and a control program for an ultrasonic diagnostic apparatus.

### Description of the Related Art

There are known ultrasonic diagnostic apparatuses that measure blood flow rates in subjects by Doppler shift in frequency of an ultrasonic echo obtained in transmission of an ultrasonic beam (e.g., see JP 2011-010789 A).

In this type of ultrasonic diagnostic apparatus, a user sets a sample gate position on a tomographic image of a subject. Then, an ultrasonic echo is selectively extracted from the sample gate position set in the tomographic image of the subject is selectively extracted, and thereby, an ultrasonic echo from blood flow in the subject and a Doppler shift in frequency thereof are detected.

At this time, the Doppler shift in frequency changes according to an intersection angle between a beam direction of an ultrasonic beam and a blood flow direction which are obtained at the sample gate position. Therefore, the Doppler shift in frequency and a blood flow rate typically have a relationship represented by the following formula (1), with the intersection angle as an angle correction value.

V=c/2 cos θ×Fd/F0 (1)

(where V: blood flow rate, F0: transmission frequency (or reception frequency) of an ultrasonic beam, Fd: Doppler shift in frequency, c: in vivo sound speed, θ: intersection angle (angle correction value))

However, as the intersection angle between a beam direction of an ultrasonic beam and a blood flow direction which are obtained at the sample gate position increases, the blood flow rate calculated on the basis of formula (1) includes an error that increases depending on an error between an actual value and set value of the intersection angle, as can be seen from formula (1). In particular, when the intersection angle exceeds 60 degrees, the error increases significantly.

Incidentally, this type of ultrasonic diagnostic apparatus is requested to have an operation content easy for the user to understand or have a reduced operation load on the user as much as possible.

### SUMMARY

Therefore, an object of the present disclosure is to provide an ultrasonic diagnostic apparatus, a control method for an ultrasonic diagnostic apparatus, and a control program for an ultrasonic diagnostic apparatus that enable highly reliable measurement of blood flow rate with reduced operation load on the user.

To achieve the abovementioned object, according to an aspect of the present invention, an ultrasonic diagnostic apparatus reflecting one aspect of the present invention comprises: a tomographic image generator that generates a tomographic image showing an inside of a subject, based on a received signal relating to an ultrasonic echo of a first ultrasonic beam transmitted to the subject; a Doppler processor that detects a Doppler shift in frequency from a transmission frequency of a second ultrasonic beam, based on a received signal relating to an ultrasonic echo of the second ultrasonic beam transmitted to the subject; a hardware processor that detects a blood vessel position in the subject based on image information about the tomographic image and sets an angle correction value relating to an intersection angle between a blood vessel extending direction at the blood vessel position and a beam direction of the second ultrasonic beam; and a display processor that generates a Doppler spectrum image representing a distribution of blood flow rate at the blood vessel position, based on the Doppler shift in frequency and the angle correction value, wherein when the intersection angle exceeds a threshold angle, the hardware processor sets the threshold angle as the angle correction value and provides notification of the setting thereof.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an appearance of an ultrasonic diagnostic apparatus according to an embodiment;

FIG. 2 is a diagram illustrating an overall configuration of an ultrasonic diagnostic apparatus according to an embodiment;

FIG. 3 is a diagram illustrating an example of a monitor screen displayed upon measurement of blood flow in an ultrasonic diagnostic apparatus according to an embodiment;

FIG. 4 is a diagram illustrating a detailed configuration of a Doppler parameter setter according to an embodiment;

FIG. 5A is a flowchart illustrating a process performed by a blood vessel detection unit according to an embodiment;

FIG. 5B is a diagram schematically illustrating a process performed by a blood vessel detection unit according to an embodiment;

FIG. 6 is a diagram schematically illustrating a process performed by a boundary detection unit according to an embodiment;

FIG. 7 is a diagram schematically illustrating a process performed by a blood vessel direction calculation unit according to an embodiment;

FIG. 8 is a diagram illustrating a notification mode performed by a first abnormality addressing unit according to an embodiment;

FIG. 9A is a diagram illustrating a guiding mode of a second abnormality addressing unit according to an embodiment;

FIG. 9B is a diagram illustrating a guiding mode of a second abnormality addressing unit according to an embodiment;

FIG. 9C is a diagram illustrating a guiding mode of a second abnormality addressing unit according to an embodiment;

FIG. 9D is a diagram illustrating a guiding mode of a second abnormality addressing unit according to an embodiment;

FIG. 9E is a diagram illustrating a guiding mode of a second abnormality addressing unit according to an embodiment;

FIG. 9F is a diagram illustrating a guiding mode of a second abnormality addressing unit according to an embodiment;

FIG. 10 is a diagram schematically illustrating a process performed by a third abnormality addressing unit according to an embodiment; and

FIG. 11 is a flowchart illustrating a series of process steps executed by a Doppler parameter setter according to an embodiment.

### DETAILED DESCRIPTION OF EMBODIMENTS

Hereinafter, one or more embodiments of the present invention will be described with reference to the drawings. However, the scope of the invention is not limited to the disclosed embodiments. In the present specification and the drawings, components having substantially the same functions are denoted by the same reference numerals, and redundant description thereof is omitted.

### Configuration of Ultrasonic Diagnostic Apparatus

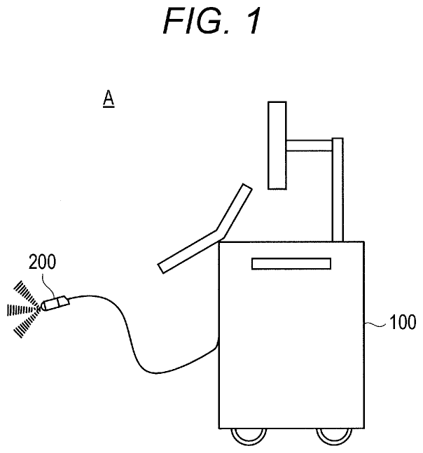

Hereinafter, a configuration of an ultrasonic diagnostic apparatus according to an embodiment of the present invention will be described with reference to FIGS. 1, 2, and 3.

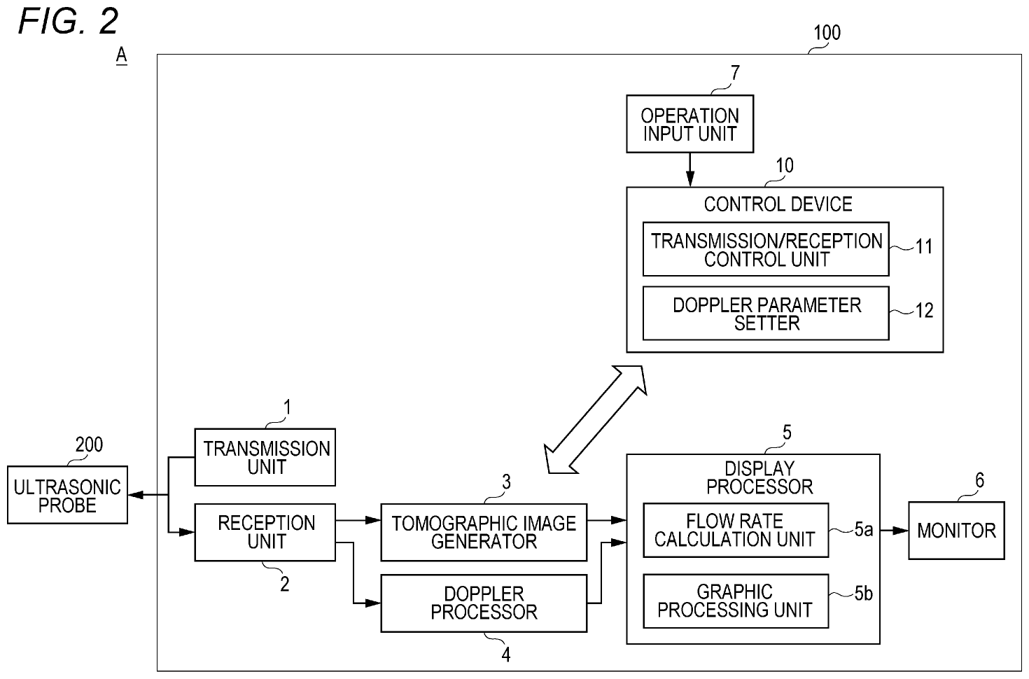

FIG. 1 is a diagram illustrating an appearance of an ultrasonic diagnostic apparatus A according to the present embodiment. FIG. 2 is a diagram illustrating an overall configuration of the ultrasonic diagnostic apparatus A according to the present embodiment.

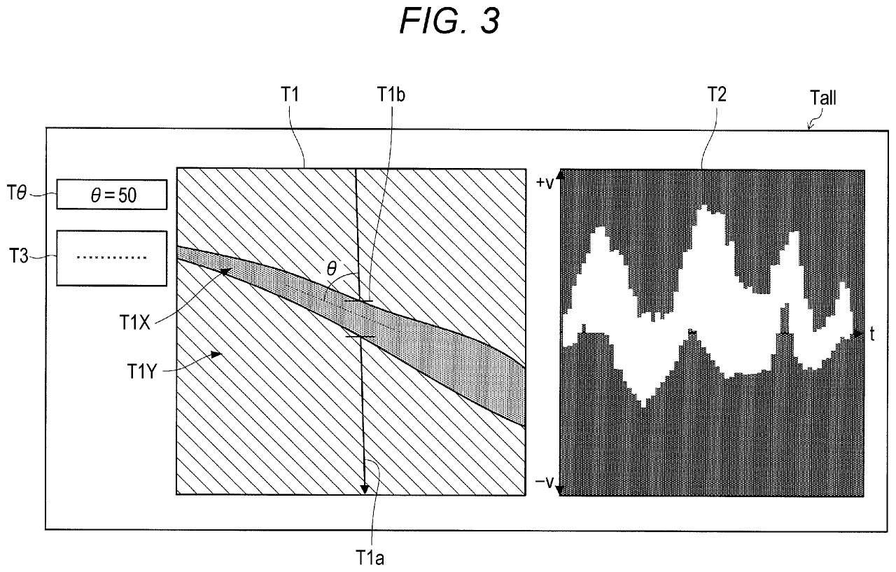

FIG. 3 is a diagram illustrating an example of a monitor screen displayed upon measurement of blood flow in the ultrasonic diagnostic apparatus A according to the present embodiment.

The ultrasonic diagnostic apparatus A is used for visualizing a shape, property, or dynamics in a subject as an ultrasonic image to perform image diagnosis. In the present embodiment, a mode in which the ultrasonic diagnostic apparatus A performs a B-mode operation and a PW Doppler mode operation in a time-division manner to generate a tomographic image and a Doppler spectrum image will be described (see FIG. 3).

As illustrated in FIG. 1, the ultrasonic diagnostic apparatus A includes an ultrasonic diagnostic apparatus body 100 and an ultrasonic probe 200. The ultrasonic diagnostic apparatus body 100 and the ultrasonic probe 200 are connected to each other via a cable.

The ultrasonic probe 200 serves as an acoustic sensor that transmits an ultrasonic beam (here, approximately 1 to 30 MHz) into a subject (e.g., a human body), receives an ultrasonic echo obtained from an ultrasonic beam reflected from inside the subject, and converts the ultrasonic echo into an electrical signal.

The user brings the ultrasonic beam transmitting/receiving surface of the ultrasonic probe 200 into contact with the subject to operate the ultrasonic diagnostic apparatus A, for ultrasonic diagnosis. Note that, here, it is intended that the ultrasonic probe 200 transmits an ultrasonic beam into the subject from an outer surface of the subject and receives an ultrasonic echo, but the ultrasonic probe 200 may be used by being inserted into a digestive tract or blood vessel, a body cavity, or the like. In addition, the ultrasonic probe 200 may include any of a convex probe, linear probe, sector probe, or three-dimensional probe.

The ultrasonic probe 200 includes, for example, a plurality of transducers (e.g., piezoelectric elements) that is arranged in a matrix, and a channel switching unit (e.g., a multiplexer) that controls on and off of a driving state of an individual or block of transducers (hereinafter, referred to as “channels”).

Each of the transducers of the ultrasonic probe 200 converts a voltage pulse generated by the ultrasonic diagnostic apparatus body 100 (transmission unit 1) into an ultrasonic beam to transmit the ultrasonic beam into the subject, receives an ultrasonic echo reflected from inside the subject to convert the received ultrasonic echo into an electric signal (hereinafter, referred to as a “received signal”), and outputs the received signal to the ultrasonic diagnostic apparatus body 100 (reception unit 2).

The ultrasonic diagnostic apparatus body 100 includes the transmission unit 1, the reception unit 2, a tomographic image generator 3, a Doppler processor 4, a display processor 5, a monitor 6, an operation input unit 7, and a control device 10.

The transmission unit 1 is a transmitter that transmits a voltage pulse as a driving signal to the ultrasonic probe 200. The transmission unit 1 includes, for example, a high-frequency pulse generator, a pulse setting unit, and the like. The transmission unit 1 adjusts the voltage pulse generated by the high-frequency pulse generator to a voltage amplitude, pulse width, and transmission timing set by the pulse setting unit and transmits the voltage pulse to each channel of the ultrasonic probe 200.

The transmission unit 1 includes the pulse setting unit for each of the plurality of channels of the ultrasonic probe 200, enabling the voltage amplitude, pulse width, and transmission timing of a voltage pulse for each of the plurality of channels to be set. For example, the transmission unit 1 changes a target depth or generates different pulse waveforms (e.g., transmits one pulse in the B mode and four pulses in the PW Doppler mode) by setting appropriate delay times for the plurality of channels.

The reception unit 2 is a receiver that receives and processes a received signal relating to an ultrasonic echo generated by the ultrasonic probe 200. The reception unit 2 includes a preamplifier, an AD conversion unit, a reception beam former, and a processing system switching unit.

The reception unit 2 amplifies a received signal relating to a weak ultrasonic echo for each channel with the preamplifier and converts the received signal into a digital signal with an AD conversion unit. Then, the reception unit 2 delays and summates received signals of the respective channels with the reception beam former to combine the received signals of the plurality of channels into one, and acoustic line data is obtained. Furthermore, the reception unit 2 performs control to switch transmission destination for a received signal generated by the reception beam former with the processing system switching unit and outputs the received signal to the tomographic image generator 3 or the Doppler processor 4 according to an operation mode to be performed.

The tomographic image generator 3 acquires a received signal from the reception unit 2 during the B-mode operation and generates a tomographic image (also referred to as a B-mode image) showing the inside of the subject.

For example, when the ultrasonic probe 200 transmits a pulsed ultrasonic beam in a depth direction, the tomographic image generator 3 temporally continuously accumulates signal intensities of ultrasonic echoes detected thereafter in a line memory. Then, the tomographic image generator 3 sequentially accumulates the signal intensities of ultrasonic echoes at scanning positions in the line memory as the ultrasonic beam from the ultrasonic probe 200 scans the inside of the subject and generates two-dimensional data as a frame unit. Then, the tomographic image generator 3 generates a tomographic image by converting the signal intensities of the ultrasonic echoes detected at the respective positions inside the subject into luminance values.

The tomographic image generator 3 includes, for example, an envelope detection circuit, a dynamic filter, and a logarithmic compression circuit. In the envelope detection circuit, a received signal is envelope detected, and signal intensity is detected. The logarithmic compression circuit performs a logarithmic compression on the signal intensity of the received signal detected by the envelope detection circuit. The dynamic filter is a band-pass filter having a frequency characteristic changed according to depth and removes a noise component included in the received signal.

The Doppler processor 4 acquires a received signal from the reception unit 2 during the PW Doppler mode operation and detects a Doppler shift in frequency with respect to a transmission frequency of an ultrasonic echo from blood flow.

The Doppler processor 4 samples a received signal relating to an ultrasonic echo in synchronization with a pulse repetition frequency while the ultrasonic probe 200 is transmitting a pulsed ultrasonic beam at regular intervals according to the pulse repetition frequency. Then, for example, the Doppler processor 4 detects a Doppler shift in frequency, on the basis of a phase difference between an ultrasonic echo relating to the nth ultrasonic beam from a sample gate position and an ultrasonic echo relating to the (n+1)th ultrasonic beam from the same sample gate position.

The Doppler processor 4 includes, for example, a quadrature detection unit, a low-pass filter, a range gate, and an FFT analysis unit. The quadrature detection unit mixes, to a received signal, a reference signal in phase with a transmitted ultrasonic beam and a reference signal out of phase with the transmitted ultrasonic beam by π/2 to generate a quadrature detection signal. The low-pass filter removes a high-frequency component of the quadrature detection signal and generates a received signal relating to the Doppler shift in frequency. The range gate acquires only ultrasonic echo from a sample gate position. The FFT analysis unit calculates the Doppler shift in frequency of an ultrasonic echo on the basis of a temporal change of a received signal output from the range gate.

The display processor 5 acquires a tomographic image output from the tomographic image generator 3 and the Doppler shift in frequency of an ultrasonic echo output from the Doppler processor 4 and generates a display image to be displayed on the monitor 6 (See FIG. 3).

The display processor 5 includes a flow rate calculation unit 5a and a graphic processing unit 5b.

The flow rate calculation unit 5a operates in the PW Doppler mode and generates a Doppler spectrum image (T2 in FIG. 3) representing a distribution of blood flow rate at a sample gate position in time-series. In the Doppler spectrum image, time is represented on the horizontal axis and blood flow rate on the vertical axis. In the Doppler spectrum image, for example, a blood flow rate at each time point is expressed in the form of a single line, and the power of each blood flow rate (i.e., each frequency) is represented by the magnitude of the luminance of a pixel (a change in luminance is not illustrated in FIG. 3).

Here, a relationship between the Doppler shift in frequency of an ultrasonic echo output from the Doppler processor 4 and a blood flow rate is expressed by the following formula (2), on the basis of an angle correction value relating to the intersection angle θ (hereinafter referred to as “beam-vessel intersection angle θ”) between a beam direction of an ultrasonic beam and an extending direction of a blood vessel. Note that the beam-vessel intersection angle θ referred to by the flow rate calculation unit 5a is set according to an instruction given from the control device 10 (Doppler parameter setter 12).

V=c/2 cos θ×Fd/F0 (2)

(where V: blood flow rate, F0: transmission frequency (or reception frequency) of an ultrasonic beam, Fd: Doppler shift in frequency, c: in vivo sound speed, θ: angle correction value)

In the generation of a Doppler spectrum image, the angle correction value is used to correct a scale value of the vertical axis of the Doppler spectral image, that is, a numerical value of the blood flow rate, on the basis of formula (2).

The graphic processing unit 5b performs predetermined image processing such as coordinate transformation processing or data interpolation processing on a tomographic image output from the tomographic image generator 3. Then, the graphic processing unit 5b combines the tomographic image subjected to the image processing and a Doppler spectrum image to generate a display image.

Furthermore, the graphic processing unit 5b previously acquires information relating to a sample gate position, sample gate size, the steering angle of an ultrasonic beam, an angle correction value, and the like set by the control device 10 (here, the Doppler parameter setter 12) and embeds an image (e.g., these numerical values and marks) corresponding to the information in the display image so that the user can perceive the information. Note that the graphic processing unit 5b typically displays an image indicating the sample gate position, sample gate size, steering angle of the ultrasonic beam, and the direction of blood flow (extending direction of a blood vessel) on the tomographic image in a superimposed manner.

The monitor screen of FIG. 3 illustrates a display image generated by the graphic processing unit 5b when the B-mode operation and the PW Doppler mode operation are being executed in parallel. In FIG. 3, Tall represents the entire region of the display image, T1 represents a tomographic image (T1X is a blood flow region, T1Y is a tissue region), T1a represents the steering angle of an ultrasonic beam in the PW Doppler mode operation, T1b represents a sample gate position for the ultrasonic beam in the PW Doppler mode operation, T2 represents a Doppler spectrum image, T3 represents an image of a message box, and TO represents an angle correction value display box indicating an angle correction value in blood flow rate calculation. Note that in the message box T3, for example, the content to be perceived by the user is displayed.

Note that the tomographic image generator 3, the Doppler processor 4, and the display processor 5 are achieved by, for example, a digital arithmetic circuit including a digital signal processor (DSP) or the like. However, these configurations can be variously modified. For example, some or all of the tomographic image generator 3, the Doppler processor 4, and the display processor 5 may be achieved by a hardware circuit or may be achieved by arithmetic processing according to a program.

The monitor 6 is a display that displays a display image generated by the display processor 5 and includes, for example, a liquid crystal display.

The operation input unit 7 is a user interface for the user to perform an input operation and includes, for example, a push button switch, a keyboard, a mouse, or the like. The operation input unit 7 converts an input operation performed by the user into an operation signal and inputs the operation signal to the control device 10.

For integrated control, the control device 10 exchanges signals with the ultrasonic probe 200, the transmission unit 1, the reception unit 2, the tomographic image generator 3, the Doppler processor 4, the display processor 5, the monitor 6, and the operation input unit 7. Note that the control device 10 includes, for example, a central processing unit (CPU), a read only memory (ROM), a random access memory (RAM), and the like. Each function of the control device 10 is achieved by the CPU referring to a control program or various data stored in the ROM and the RAM. However, as a matter of course, some or all of the functions of the control device 10 may be achieved by dedicated hardware circuits or a combination thereof, including but not limited to processing by software.

The control device 10 includes a transmission/reception control unit 11 and the Doppler parameter setter 12.

The transmission/reception control unit 11 causes the channel switching unit (not illustrated) of the ultrasonic probe 200 to selectively determine channels to be driven from among the plurality of channels. Then, the transmission/reception control unit 11 causes each of the transmission unit 1 and the reception unit 2 to perform transmission and reception of an ultrasonic wave to and from the channels to be driven.

The transmission/reception control unit 11 sequentially drives the channels to be driven of the plurality of channels along a scanning direction during the B-mode operation (i.e., when generating a tomographic image) and causes the ultrasonic probe 200 to perform ultrasonic scanning in the subject.

In the PW Doppler mode operation (i.e., when measuring blood flow rate), the transmission/reception control unit 11 causes the plurality of transducers provided in the ultrasonic probe 200 to be selectively driven so as to transmit an ultrasonic beam from the ultrasonic probe 200 to a sample gate position in the subject at a predetermined angle. Furthermore, at this time, the transmission/reception control unit 11 controls the transmission unit 1 so that a pulsed ultrasonic beam (burst wave) may be repeatedly transmitted from the ultrasonic probe 200 at a predetermined pulse repetition frequency and controls the reception unit 2 so as to receive an ultrasonic echo of the ultrasonic beam.

The transmission/reception control unit 11 basically determines ultrasonic beam transmission/reception conditions, on the basis of the type of the ultrasonic probe 200 (e.g., complex type, sector type, or linear type), the depth of an imaging target in the subject, and an imaging mode (e.g., B-mode, PW Doppler mode, or M-mode) and the like, which are set by the user via the operation input unit 7.

However, for the PW Doppler mode operation, the transmission/reception control unit 11 determines the ultrasonic beam transmission/reception conditions, on the basis of a sample gate position, sample gate size, and a beam direction of an ultrasonic beam (i.e., steering angle), which are set in the Doppler parameter setter 12.

For the PW Doppler mode operation, the Doppler parameter setter 12 sets various parameters so as to detect the flow rate of blood through a blood vessel in the subject. Typically, the Doppler parameter setter 12 automatically sets the sample gate position, sample gate size, steering angle of an ultrasonic beam, and angle correction value that is referred to in formula (2), on the basis of image information about a tomographic image.

However, the Doppler parameter setter 12 may have a function of setting the sample gate position, sample gate size, steering angle of an ultrasonic beam, and angle correction value by the user's manual operation, in addition to the automatic setting thereof.

### Detailed Configuration of Doppler Parameter Setter 12

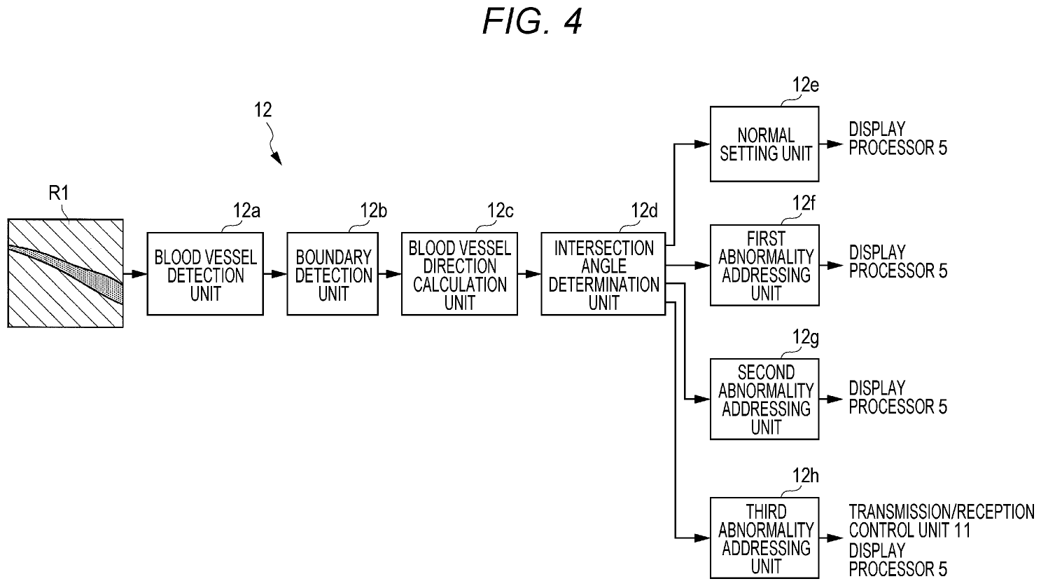

Next, a detailed configuration of the Doppler parameter setter 12 will be described with reference to FIGS. 4 to 10.

FIG. 4 is a diagram illustrating a detailed configuration of the Doppler parameter setter 12 according to the present embodiment.

The Doppler parameter setter 12 includes a blood vessel detection unit 12a, a boundary detection unit 12b, a blood vessel direction calculation unit 12c, an intersection angle determination unit 12d, a normal setting unit 12e, a first abnormality addressing unit 12f, a second abnormality addressing unit 12g, and a third abnormality addressing unit 12h.

### Blood Vessel Detection Unit 12a

The blood vessel detection unit 12a acquires a tomographic image R1 generated by the tomographic image generator 3 and detects a position of a blood vessel shown in the tomographic image R1 on the basis of image information about the tomographic image R1. The blood vessel detection unit 12a detects the position of a blood vessel shown in the tomographic image R1 with blood vessel pattern data (i.e., template image) stored in a memory (not illustrated) in advance, for example, by known template matching.

Then, the blood vessel detection unit 12a sets, for example, a region of the tomographic image R1 where the blood vessel is most clearly shown as a sample gate position to be subjected to Doppler processing (i.e., a center position of the sample gate).

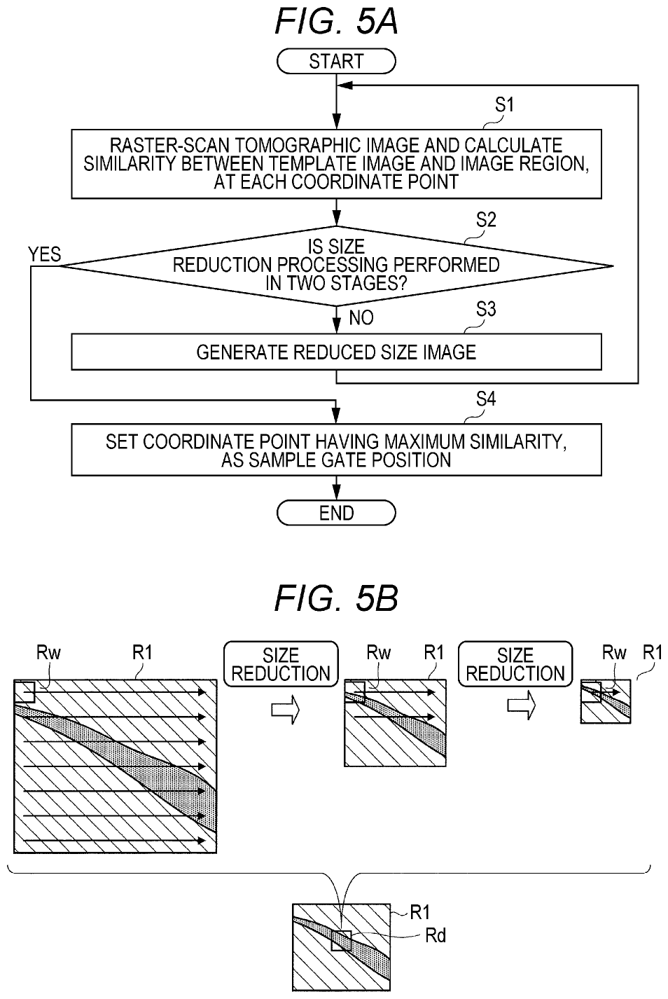

FIG. 5A is a flowchart illustrating a process performed by the blood vessel detection unit 12a according to the present embodiment. FIG. 5B is a diagram schematically illustrating a process performed by the blood vessel detection unit 12a according to the present embodiment.

Firstly, in step S1, the blood vessel detection unit 12a reads a template image of the blood vessel. Then, the blood vessel detection unit 12a, for example, sequentially sets comparison target image regions (hereinafter referred to as “comparison target regions”) of the same size (e.g., 100 pixels×100 pixels) as a template image Rw in the tomographic image R1, in a raster scan manner in the tomographic image R1 and calculates similarity with the template image Rw for each of the comparison target regions. Then, the blood vessel detection unit 12a calculates similarities with the template image Rw at each coordinate point in the tomographic image R1.

In this way, a region where the blood vessel is clearly shown is searched for in the tomographic image R1.

Next, in step S2, the blood vessel detection unit 12a determines whether size reduction processing in subsequent step S3 has been performed in two stages. If the size reduction processing in step S3 has been performed in two stages (step S2: YES), the process proceeds to step S4, and if the size reduction processing in step S3 has not been performed in two stages (step S2: NO), the process proceeds to step S3.

Next, in step S3, the blood vessel detection unit 12a reduces the tomographic image R1 by a predetermined magnification (e.g., 0.9 times) to generate a reduced size image. Then, returning to step S1, the blood vessel detection unit 12a similarly performs template matching on the reduced size image similarly by using the template image of the blood vessel and calculates similarity for each coordinate point of the reduced size image. In this case, the template image of the blood vessel applied to the original tomographic image R1 is used without changing the size of the template image of the blood vessel.

Note that this search processing using the reduced size image is processing in consideration of the size of the blood vessel that is different between the tomographic image and the template image.

Next, in step S4, the blood vessel detection unit 12a selects a coordinate point having a maximum similarity from the coordinate points of the tomographic image R1, coordinate points of the reduced size image, and coordinate points of a further reduced size image (tomographic image R1 reduced in size in two stages).

In such processing, the blood vessel detection unit 12a searches for a region Rd where the blood vessel is most clearly shown in the tomographic image R1 and sets the region (i.e., center coordinates) Rd as a sample gate position (i.e., a center position of the sample gate).

Note that any method of detecting a blood vessel by the blood vessel detection unit 12a may be employed, and a discriminator (e.g., CNN) or the like that has been learned by machine learning may be used.

### Boundary Detection Unit 12b

The boundary detection unit 12b detects a boundary position (i.e., blood vessel wall) between the blood vessel and extravascular tissue, in an area around coordinates set as the sample gate position in the tomographic image R1 by the blood vessel detection unit 12a. Then, the boundary detection unit 12b sets a sample gate size on the basis of the boundary position.

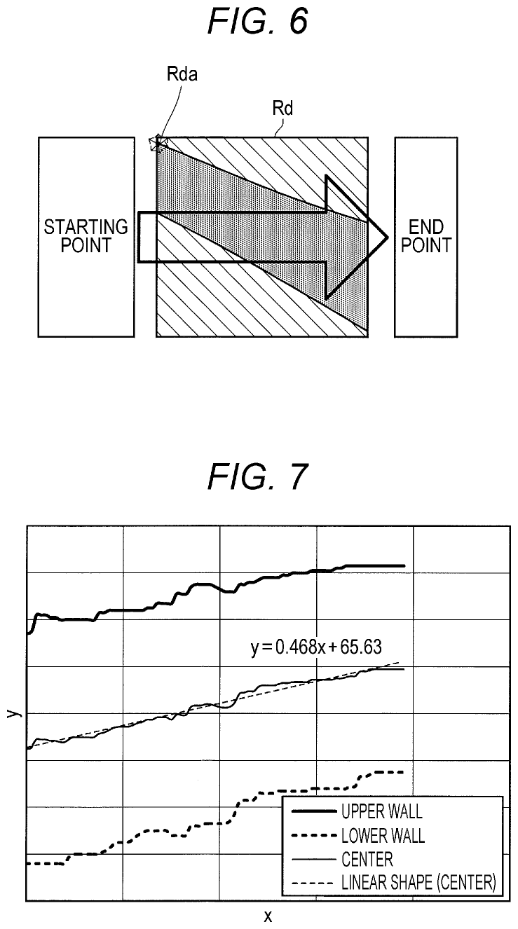

FIG. 6 is a diagram schematically illustrating a process performed by a boundary detection unit 12b according to the present embodiment. For example, in the image region Rd around the coordinates set as the sample gate position, the boundary detection unit 12b regards a path having an edge strong and smoothly continued as a boundary between the blood vessel and extravascular tissue and searches for the path. Specifically, in the boundary detection unit 12b, boundary detection is regarded as a path search that searches for a path having the minimum cost, that is, a direction in which the strength of the edge reduces and a direction in which the path has no smoothness is regarded as a direction in which the cost increases, and a path having the minimum cost is searched for from the left end side (Rda in FIG. 6) of the image region Rd. Thus, a boundary position between an upper side wall portion of the blood vessel and extravascular tissue and a boundary position between a lower side wall portion of the blood vessel and the extravascular tissue are detected. Then, the boundary detection unit 12b sets the width between the boundary position of the upper side wall portion of the blood vessel and the boundary position of the lower side wall portion of the blood vessel as the sample gate size.

Note that information relating to the sample gate position set by the blood vessel detection unit 12a and sample gate size set by the boundary detection unit 12b is transmitted to the transmission/reception control unit 11, as ultrasonic beam transmission/reception conditions during the PW Doppler mode operation.

### Blood Vessel Direction Calculation Unit 12c

The blood vessel direction calculation unit 12c calculates an extending direction of the blood vessel at the sample gate position, on the basis of the boundary position of the blood vessel detected by the boundary detection unit 12b.

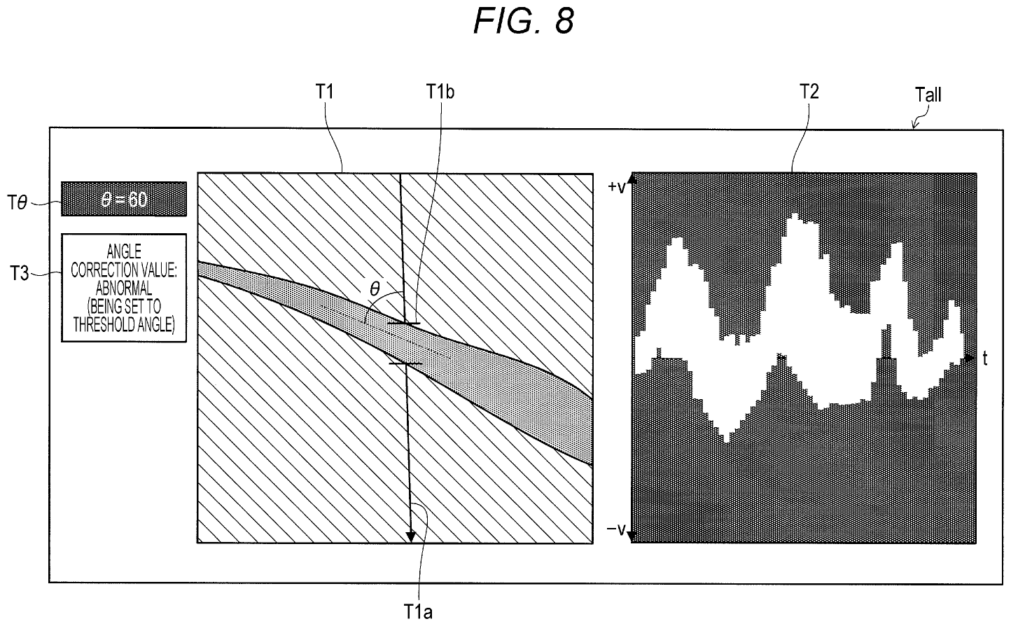

FIG. 7 is a diagram schematically illustrating a process performed by the blood vessel direction calculation unit 12c according to the present embodiment. The blood vessel direction calculation unit 12c calculates, as the extending direction of the blood vessel, for example, an average value of an extending direction of the boundary of the upper side wall portion of the blood vessel and an extending direction of the boundary of the lower side wall portion of the blood vessel. Note that in FIG. 7, the extending direction of the blood vessel is calculated as tilt angle of an XY coordinate system, taking a scanning direction in the tomographic image R1 on the X-axis and taking a depth direction thereof on the Y-axis.

### Intersection Angle Determination Unit 12d

On the basis of a set beam direction of an ultrasonic beam and the extending direction of the blood vessel calculated by the blood vessel direction calculation unit 12c, the intersection angle determination unit 12d calculates the beam-vessel intersection angle θ (intersection angle θ between the extending direction of the blood vessel and the beam direction of the ultrasonic beam, at the sample gate position). Then, the intersection angle determination unit 12d determines whether the beam-vessel intersection angle θ exceeds a threshold angle.

Here, the angle of a boundary at which an error in the blood flow rate becomes extremely large on the basis of formula (2), for example, 60 degrees is set as the threshold angle of the beam-vessel intersection angle θ. Note that as the beam direction of the ultrasonic beam that is referred to when calculating the beam-vessel intersection angle θ, for example, a steering angle set in advance by the user is used.

Note that, here, when the calculated beam-vessel intersection angle θ is equal to or smaller than the threshold angle, the Doppler parameter setter 12 performs processing in the normal setting unit 12e, and when the calculated beam-vessel intersection angle θ exceeds the threshold angle, the Doppler parameter setter 12 performs processing in the first abnormality addressing unit 12f, the second abnormality addressing unit 12g, and the third abnormality addressing unit 12h.

### Normal Setting Unit 12e

The normal setting unit 12e functions when the beam-vessel intersection angle θ is equal to or smaller than the threshold angle. The normal setting unit 12e acquires the beam-vessel intersection angle θ calculated by the intersection angle determination unit 12d, directly sets the beam-vessel intersection angle θ as the angle correction value, and indicates the set value to the display processor 5 (flow rate calculation unit 5a).

### First Abnormality Addressing Unit 12f

The first abnormality addressing unit 12f functions when the beam-vessel intersection angle θ exceeds the threshold angle. The first abnormality addressing unit 12f sets the threshold angle (e.g., 60 degrees) used for determination by the intersection angle determination unit 12d, as the angle correction value, and indicates the set value to the display processor 5 (flow rate calculation unit 5a). Furthermore, the first abnormality addressing unit 12f, for example, instructs the display processor 5 (graphic processing unit 5b) to notify the user of the settings by inverting color, changing letter color, blinking, displaying a message, or the like in a display screen on the monitor 6.

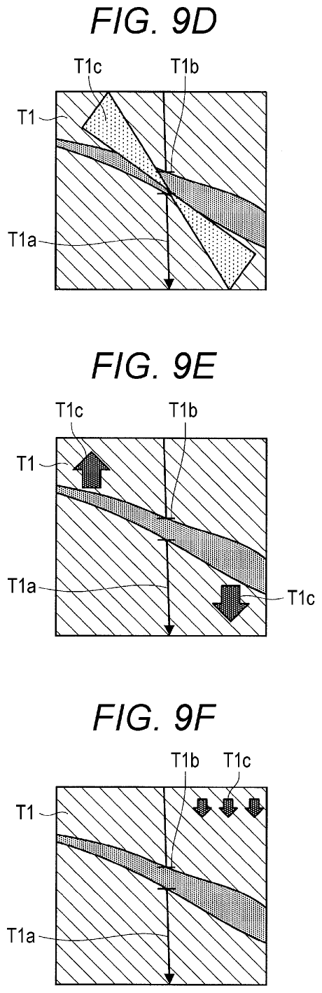

Note that, here, the first abnormality addressing unit 12f is configured to set the angle correction value to the value of the threshold angle (here, 60 degrees). This is because in a case where the beam-vessel intersection angle θ exceeds the threshold angle, the beam-to-vessel intersection angle θ is caused to be immediately returned to the threshold angle (described later with reference to FIG. 9A) in response to a notification, operation guide, and the like to the user. This makes it possible to reduce an operation load for resetting the angle correction value.

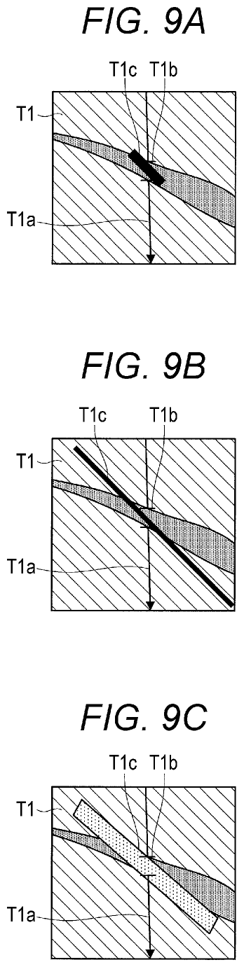

FIG. 8 is a diagram illustrating a notification mode performed by the first abnormality addressing unit 12f according to the present embodiment. The first abnormality addressing unit 12f transmits, for example, a display change instruction to the display processor 5 and causes the display processor 5 to display an image (e.g., “ANGLE CORRECTION VALUE: ABNORMAL (BEING SET TO THRESHOLD ANGLE)”) indicating that the angle correction value is being set to the threshold angle, in the message box T3 on the monitor 6.

### Second Abnormality Addressing Unit 12g

The second abnormality addressing unit 12g functions when the beam-vessel intersection angle θ exceeds the threshold angle. The second abnormality addressing unit 12g informs the user of an operation content for setting the beam-vessel intersection angle θ to or below the threshold angle through a display content in the display screen on the monitor 6.

The operation content informed of by the second abnormality addressing unit 12g is typically a guide to changing the attitude of the ultrasonic probe 200. However, the second abnormality addressing unit 12g may provide information of a guide to a steering angle changing operation of changing the steering angle of an ultrasonic beam, instead of or in addition to the guide to changing the attitude of the ultrasonic probe 200.

FIG. 9A is a diagram illustrating guiding mode of the second abnormality addressing unit 12g according to the present embodiment. The second abnormality addressing unit 12g transmits, for example, a display instruction for displaying an image for operation guide to the display processor 5 (graphic processing unit 5b) to cause the monitor 6 to display an image (hereinafter, referred to as “guide mark image”) T1c that provides information of changing the attitude of the ultrasonic probe 200, so as to be superimposed on the tomographic image T1.

The guide mark image T1c enables, for example, a difference between a current beam-vessel intersection angle θ and the threshold angle (here, 60 degrees) to be perceived at the sample gate position. The guide mark image T1c in FIG. 9A is a line image indicating a beam direction of an ultrasonic beam in which the beam-vessel intersection angle θ is to be set to the threshold angle, the guide mark image T1c being superimposed on a set sample gate position.

A mark is printed on the ultrasonic probe 200 to allow the user to discriminate one side from the other side in the scanning direction, and the user can visually perceive the guide mark image T1c displayed on the tomographic image T1 in a superimposed manner. This makes it possible for the user to understand that the attitude of the ultrasonic probe 200 should be changed in which direction and how much. Note that FIG. 9A shows that the attitude of the ultrasonic probe 200 needs to be tilted so that the beam direction of the ultrasonic beam may be tilted to the left side, in order to set the beam-vessel intersection angle θ to the threshold angle.

FIGS. 9B to 9F each illustrate another example of the guide mark image T1c. In each of FIGS. 9B, 9C, and 9D, the guide mark image T1c is illustrated as another mode of the line image indicating the beam direction of the ultrasonic beam in which the beam-vessel intersection angle θ is to be set to the threshold angle. Each of FIGS. 9E and 9F illustrates the guide mark image T1c as an arrow mark image and shows a mode in which an operation content for changing the attitude of the ultrasonic probe 200 is more directly suggested.

Note that, in order to more effectively exert the function of the second abnormality addressing unit 12g, it is preferable for the Doppler parameter setter 12 to sequentially detect the beam-vessel intersection angle θ on the basis of tomographic images R1 continuously generated by the tomographic image generator 3. Thus, the second abnormality addressing unit 12g is configured to sequentially change the information about operation content on the basis of a beam-vessel intersection angle θ detected.

### Third Abnormality Addressing Unit 12h

The third abnormality addressing unit 12h functions when the beam-vessel intersection angle θ exceeds the threshold angle. The third abnormality addressing unit 12h automatically changes the steering angle of the ultrasonic beam so that the beam-vessel intersection angle θ may be equal to or smaller than the threshold.

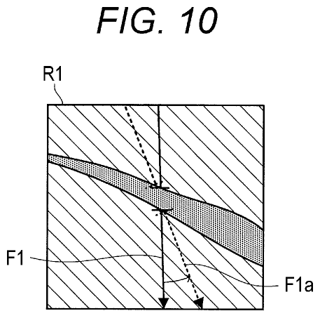

FIG. 10 is a diagram schematically illustrating a process performed by the third abnormality addressing unit 12h according to the present embodiment. In FIG. 10, F1 represents a beam direction of the ultrasonic beam before changing, and F1a represents a beam direction of the ultrasonic beam after changing.

The third abnormality addressing unit 12h, for example, determines the steering angle of the ultrasonic beam, on the basis of a changeable range of the steering angle of the ultrasonic beam, a current steering angle of the ultrasonic beam, and the extending direction of the blood vessel at the sample gate position. The steering angle of the ultrasonic beam is determined so that the beam-vessel intersection angle θ may be reduced as much as possible. Then, the third abnormality addressing unit 12h transmits a steering angle change instruction to the transmission/reception control unit 11 to have the determined beam direction. Then, the third abnormality addressing unit 12h sets a beam-vessel intersection angle θ calculated from the changed steering angle as the angle correction value and indicates the set value to the display processor 5 (flow rate calculation unit 5a).

Furthermore, when the steering angle of the ultrasonic beam is changed, the third abnormality addressing unit 12h transmits a display instruction for displaying a notification image to the display processor 5 (graphic processing unit 5b) and notifies the user of the change of the steering angle of the ultrasonic beam by inverting color, changing letter color, blinking, displaying a message, or the like in a display screen on the monitor 6.

Here, in a case where the beam-vessel intersection angle θ cannot be set to or below the threshold angle within the changeable range of the steering angle of the ultrasonic beam, the third abnormality addressing unit 12h sets the angle correction value to the value of the threshold angle, as in the first abnormality addressing unit 12f. Furthermore, in this case, the third abnormality addressing unit 12h notifies the user of setting of the angle correction value to the value of the threshold angle by inverting color, changing letter color, blinking, displaying a message, or the like in a display screen on the monitor 6.

Note that when the steering angle change instruction is given from the third abnormality addressing unit 12h, the transmission/reception control unit 11 changes the number of a drive target channel used in the PW Doppler mode, a delay time in each channel, and the like to change the steering angle while maintaining the sample gate position.

### Operation Process of Doppler Parameter Setter 12

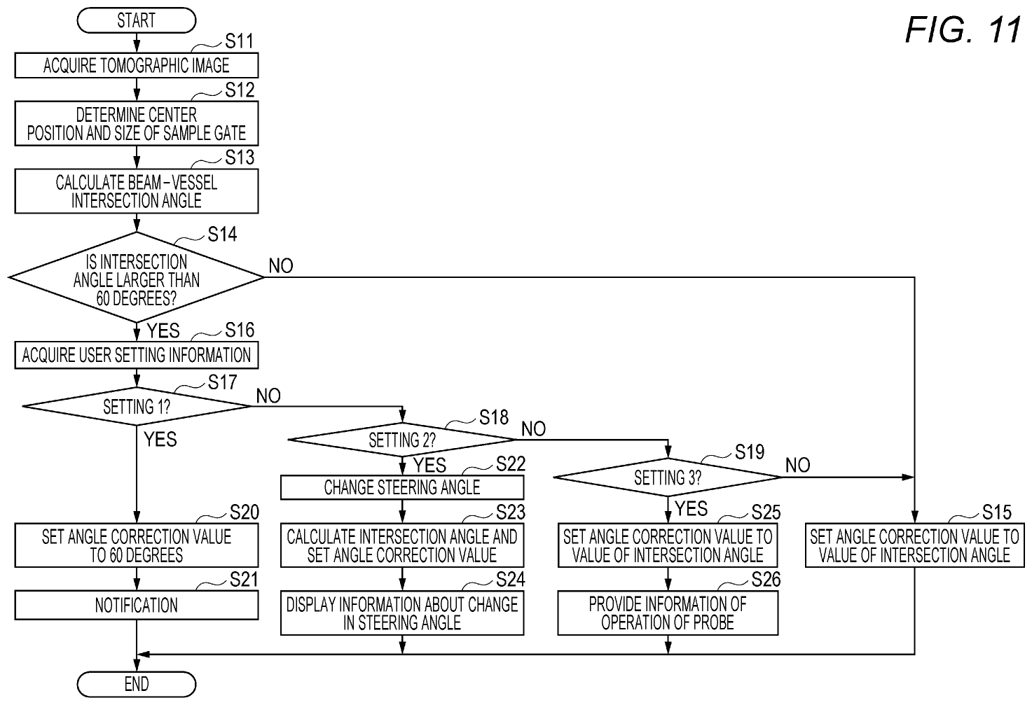

Next, an example of an operation process of the Doppler parameter setter 12 will be described with reference to FIG. 11

FIG. 11 is a flowchart illustrating a series of process steps executed by the Doppler parameter setter 12 according to the present embodiment. The flowchart illustrated in FIG. 11 is executed, for example, by the control device 10 (Doppler parameter setter 12) according to a computer program on the basis of a user's execution command for execution of the PW Doppler mode.

Here, the Doppler parameter setter 12 is configured to cause the user to selectively set processing responding to the beam-vessel intersection angle θ that exceeds the threshold angle (here, 60 degrees). The processing is selected from three modes (corresponding to “setting 1,” “setting 2,” and “setting 3” in FIG. 11) that are performed by the first abnormality addressing unit 12f, the second abnormality addressing unit 12g, and the third abnormality addressing unit 12h. This makes it possible to perform processing meeting individual users' requests, for example, for fixing the attitude of the ultrasonic probe 200 and for fixing the steering angle of the ultrasonic beam.

Firstly, in step S11, the Doppler parameter setter 12 acquires a tomographic image.

Next, in step S12, the Doppler parameter setter 12 (the blood vessel detection unit 12a, the boundary detection unit 12b, and the blood vessel direction calculation unit 12c) detects a position of a blood vessel, a boundary between the blood vessel and extravascular tissue, and an extending direction of the blood vessel, on the basis of image information about the tomographic image. Then, the Doppler parameter setter 12 sets the sample gate position and the sample gate size on the basis of the position of a blood vessel, boundary between the blood vessel and extravascular tissue, and extending direction of the blood vessel.

Next, in step S13, the Doppler parameter setter 12 (intersection angle determination unit 12d) calculates the beam-vessel intersection angle θ, on the basis of the extending direction of the blood vessel at the sample gate position detected in step S12 and a beam direction of the ultrasonic beam set by the user.

Next, in step S14, the Doppler parameter setter 12 (intersection angle determination unit 12d) determines whether the beam-vessel intersection angle θ is larger than 60 degrees. If the beam-vessel intersection angle θ is larger than 60 degrees (S14: YES), the process proceeds to step S16, and if the beam-vessel intersection angle θ is equal to or smaller than 60 degrees (S14: NO), the process proceeds to step S15.

Here, in step S15, the Doppler parameter setter 12 (normal setting unit 12e) sets an angle correction value to the value of the beam-vessel intersection angle θ.

On the other hand, in step S16, the Doppler parameter setter 12 acquires user setting information set as processing responding to the beam-vessel intersection angle θ that exceeds the threshold angle (here, 60 degrees). Then, in steps S17 to S19, the Doppler parameter setter 12 determines which of the above “setting 1” to “setting 3” is set. Specifically, in step S17, the Doppler parameter setter 12 determines whether setting 1” is set. If “setting 1” is set (S17: YES), the process proceeds to step S20, and if “setting 1” is not set (S17: NO), the process proceeds to step S18. Then, in step S18, the Doppler parameter setter 12 determines whether “setting 2” is set. If “setting 2” is set (S18: YES), the process proceeds to step S22, and if “setting 2” is not set (S18: NO), the process proceeds to step S19. Then, in step S19, the Doppler parameter setter 12 determines whether “setting 3” is set. If “setting 3” is set (S19: YES), the process proceeds to step S25, and if “setting 3” is not set (S19: NO), the process proceeds to step S15.

In step S20 (if “setting 1” is set), the Doppler parameter setter 12 (first abnormality addressing unit 12f) sets the angle correction value to the value of the threshold angle regardless of the beam-vessel intersection angle θ. Then, in step S21, the Doppler parameter setter 12 (first abnormality addressing unit 12f) notifies that the angle correction value is set to the value of the threshold angle in the message box T3 or the like displayed on the monitor 6 (e.g., see FIG. 8).

Note that, at this time, the user looks at display in the message box T3 displayed on the monitor 6 and perceives that the beam-vessel intersection angle θ exceeds the threshold angle. Then, for example, the user performs an operation to change the attitude of the ultrasonic probe 200 or the steering angle of the ultrasonic beam to reduce the beam-vessel intersection angle θ to the threshold angle. The operation is performed while watching the images of the steering angle T1a of the ultrasonic beam and the sample gate position T1b for the ultrasonic beam that are indicated on the tomographic image T1 displayed on the monitor 6, and the image of the blood flow region T1X that is displayed in the tomographic image T1. Thus, the blood flow rate is measured in a state where the beam-vessel intersection angle θ is reduced to the threshold angle.

In step S22 (if “setting 2” is set), the Doppler parameter setter 12 (third abnormality addressing unit 12h) changes the steering angle of the ultrasonic beam to reduce the beam-vessel intersection angle θ as much as possible (e.g., see FIG. 10). Then, in step S23, on the basis of the changed steering angle, the Doppler parameter setter 12 (second abnormality addressing unit 12g) calculates the beam-vessel intersection angle θ again and sets the angle correction value. Then, in step S24, the Doppler parameter setter 12 (second abnormality addressing unit 12g) displays information about the changing of the steering angle in the message box T3 or the like displayed on the monitor 6.

Note that, at this time, if the beam-vessel intersection angle θ is equal to or smaller than the threshold angle, the user does not need to perform any operation. However, if the beam-vessel intersection angle θ exceeds the threshold angle even after the steering angle is changed, the angle correction value is set to the value of the threshold angle, and the user performs an operation to change the attitude of the ultrasonic probe 200. Thus, the blood flow rate is measured in a state where the beam-vessel intersection angle θ is reduced to the threshold angle.

In step S25 (if “setting 3” is set), the Doppler parameter setter 12 (second abnormality addressing unit 12g) sets the angle correction value to the value of the threshold angle. Then, in step S26, the Doppler parameter setter 12 (second abnormality addressing unit 12g) provides information of changing the attitude of the ultrasonic probe 200 or changing the steering angle of the ultrasonic beam (e.g., see FIG. 9A). The changing is necessary to set the beam-vessel intersection angle θ to or below the threshold angle.

Note that, at this time, the user performs an operation of changing the attitude of the ultrasonic probe 200 or operation of changing the steering angle of the ultrasonic beam while watching the guide mark image T1c displayed on the monitor 6. Thus, the blood flow rate is measured in a state where the beam-vessel intersection angle θ is reduced to the threshold angle.

Through a series of processing steps as described above, the Doppler parameter setter 12 appropriately sets parameters (e.g., the sample gate position, the sample gate size, and the angle correction value) relating to the sample gate in the Doppler processing, for highly reliable blood flow rate measurement.

### Effects

As described above, according to the ultrasonic diagnostic apparatus A (Doppler parameter setter 12) according to the present embodiment, parameters (e.g., sample gate position, sample gate size, and angle correction value) relating to the sample gate in Doppler processing can be automatically set so that blood flow rate can be measured with high accuracy. Then, when the beam-vessel intersection angle θ exceeds the threshold angle, the Doppler parameter setter 12 causes the first abnormality addressing unit 12f, the second abnormality addressing unit 12g, or the third abnormality addressing unit 12h to function, setting the beam-vessel intersection angle θ to or below the threshold angle.

This makes it possible to measure blood flow rate with high reliability while reducing an operation load on the user.

Note that, in the above embodiment, for an example of the Doppler parameter setter 12, when the beam-vessel intersection angle θ exceeds the threshold angle, only one that is set by the user from the first abnormality addressing unit 12f, the second abnormality addressing unit 12g, and the third abnormality addressing units 12h functions, but all or two of the abnormality addressing units may function.

In particular, with only the first abnormality addressing unit 12f, it may be difficult for an unskilled user to understand how to reduce the beam-vessel intersection angle θ. Therefore, It is desirable to cause both of the first abnormality addressing unit 12f and the second abnormality addressing unit 12g to function. On the other hand, from the viewpoint of simplification in configuration of the ultrasonic diagnostic apparatus A, the Doppler parameter setter 12 may include only one of the first abnormality addressing unit 12f, the second abnormality addressing unit 12g, and the third abnormality addressing unit 12h.

Furthermore, in the above-described embodiment, for an example of a mode of notifying or informing the user of the Doppler parameter setter 12 of information, the color invert, character color change, blinking, or message display on the display screen of the monitor 6 has been described. However, for a mode in which the Doppler parameter setter 12 notifies or informs the user of information, warning sound given by a speaker or another device may be used.

Furthermore, in the above embodiment, the PW Doppler mode has been described as an example of a target to which the Doppler parameter setter 12 is applied. However, the configuration of the Doppler parameter setter 12 is also applicable to the operation of the ultrasonic diagnostic apparatus A in the CW Doppler mode.

According to an ultrasonic diagnostic apparatus according to the present disclosure, it is possible to perform highly reliable measurement of blood flow rate with reduced operation load on the user.

Although embodiments of the present invention have been described and illustrated in detail, the disclosed embodiments are made for purposes of illustration and example only and not limitation. The scope of the present invention should be interpreted by terms of the appended claims The technique described in the claims includes various modification and alterations of the above specific embodiments.

What is claimed is:

1. An ultrasonic diagnostic apparatus comprising:
a tomographic image generator that generates a tomographic image showing an inside of a subject, based on a received signal relating to an ultrasonic echo of a first ultrasonic beam transmitted to the subject;
a Doppler processor that detects a Doppler shift in frequency from a transmission frequency of a second ultrasonic beam, based on a received signal relating to an ultrasonic echo of the second ultrasonic beam transmitted to the subject;
a hardware processor that detects a blood vessel position in the subject based on image information about the tomographic image and sets an angle correction value relating to an intersection angle between a blood vessel extending direction at the blood vessel position and a beam direction of the second ultrasonic beam; and
a display processor that generates a Doppler spectrum image representing a distribution of blood flow rate at the blood vessel position, based on the Doppler shift in frequency and the angle correction value,
wherein when the intersection angle exceeds a threshold angle, the hardware processor sets the threshold angle as the angle correction value and provides notification of the setting thereof.

2. The ultrasonic diagnostic apparatus according to claim 1, wherein
the hardware processor performs notification by at least one of color inversion, character color change, blinking or message display in a display screen on a monitor, and a warning sound from a speaker.

3. The ultrasonic diagnostic apparatus according to claim 1, wherein
the hardware processor notifies that the intersection angle exceeds the threshold angle.

4. The ultrasonic diagnostic apparatus according to claim 1, wherein
the hardware processor, when the intersection angle exceeds the threshold angle, informs a user of an operation content for setting the intersection angle to or below the threshold angle.

5. The ultrasonic diagnostic apparatus according to claim 4, wherein
the operation content is a guide to changing the attitude of an ultrasonic probe that transmits the second ultrasonic beam or a guide to a steering angle changing operation of changing the beam direction of the second ultrasonic beam.

6. The ultrasonic diagnostic apparatus according to claim 1, wherein
the hardware processor, when the intersection angle is equal to or smaller than a threshold angle, sets the intersection angle as the angle correction value.

7. The ultrasonic diagnostic apparatus according to claim 1, wherein
the hardware processor sets a sample gate size at a sample gate position based on image information about the tomographic image.

8. The ultrasonic diagnostic apparatus according to claim 1, wherein
the hardware processor sets, as a sample gate position, a blood vessel position in the subject detected based on image information about the tomographic image.

9. An ultrasonic diagnostic apparatus comprising:
a tomographic image generator that generates a tomographic image showing an inside of a subject, based on a received signal relating to an ultrasonic echo of a first ultrasonic beam transmitted to the subject;
a Doppler processor that detects a Doppler shift in frequency from a transmission frequency of a second ultrasonic beam, based on a received signal relating to an ultrasonic echo of the second ultrasonic beam transmitted to the subject;
a hardware processor that detects a blood vessel position in the subject based on image information about the tomographic image and sets an angle correction value relating to an intersection angle between a blood vessel extending direction at the blood vessel position and a beam direction of the second ultrasonic beam; and
a display processor that generates a Doppler spectrum image representing a distribution of blood flow rate at the blood vessel position, based on the Doppler shift in frequency and the angle correction value,
wherein the hardware processor, when the intersection angle exceeds the threshold angle, changes the beam direction of the second ultrasonic beam so that the intersection angle may be equal to or smaller than the threshold angle.

10. The ultrasonic diagnostic apparatus according to claim 9, wherein
the hardware processor, when the beam direction of the second ultrasonic beam is changed, provides notification of changing of the beam direction of the second ultrasonic beam, by color inversion, character color change, blinking or message display in a display screen on a monitor, or a warning sound from a speaker.

11. The ultrasonic diagnostic apparatus according to claim 9, wherein
in a case where the intersection angle cannot be reduced to or below the threshold angle within a range where the beam direction of the second ultrasonic beam can be changed, the hardware processor sets the threshold angle as the angle correction value and notifies a user of setting of the threshold angle as the angle correction value.

12. An ultrasonic diagnostic apparatus comprising:
a tomographic image generator that generates a tomographic image showing an inside of a subject, based on a received signal relating to an ultrasonic echo of a first ultrasonic beam transmitted to the subject;
a Doppler processor that detects a Doppler shift in frequency from a transmission frequency of a second ultrasonic beam, based on a received signal relating to an ultrasonic echo of the second ultrasonic beam transmitted to the subject;
a hardware processor that detects a blood vessel position in the subject based on image information about the tomographic image and sets an angle correction value relating to an intersection angle between a blood vessel extending direction at the blood vessel position and a beam direction of the second ultrasonic beam; and
a display processor that generates a Doppler spectrum image representing a distribution of blood flow rate at the blood vessel position, based on the Doppler shift in frequency and the angle correction value,
wherein the hardware processor, when the intersection angle exceeds the threshold angle, informs a user of an operation content of setting the intersection angle to or below the threshold angle.

13. The ultrasonic diagnostic apparatus according to claim 12, wherein
the hardware processor, when the intersection angle exceeds the threshold angle, notifies the user that the intersection angle exceeds the threshold angle, by color inversion, character color change, blinking or message display in a display screen on a monitor, or a warning sound from a speaker.

14. The ultrasonic diagnostic apparatus according to claim 12, wherein
the operation content is a guide to changing the attitude of an ultrasonic probe that transmits the second ultrasonic beam or a guide to a steering angle changing operation of changing the beam direction of the second ultrasonic beam.

15. The ultrasonic diagnostic apparatus according to claim 12, wherein
the hardware processor sequentially detects the intersection angle based on the tomographic images continuously generated by the tomographic image generator and changes the operation content based on the intersection angle detected.

16. The ultrasonic diagnostic apparatus according to claim 12, wherein
the hardware processor causes a monitor to display the operation content so as to be superimposed on the tomographic image.

17. The ultrasonic diagnostic apparatus according to claim 12, wherein
the operation content is an image that enables perception of a difference between a current intersection angle and the threshold angle.

18. An ultrasonic diagnostic apparatus comprising:
a tomographic image generator that generates a tomographic image showing an inside of a subject, based on a received signal relating to an ultrasonic echo of a first ultrasonic beam transmitted to the subject;
a Doppler processor that detects a Doppler shift in frequency from a transmission frequency of a second ultrasonic beam, based on a received signal relating to an ultrasonic echo of the second ultrasonic beam transmitted to the subject;
a hardware processor that detects a blood vessel position in the subject based on image information about the tomographic image and sets an angle correction value relating to an intersection angle between a blood vessel extending direction at the blood vessel position and a beam direction of the second ultrasonic beam; and
a display processor that generates a Doppler spectrum image representing a distribution of blood flow rate at the blood vessel position, based on the Doppler shift in frequency and the angle correction value,
wherein the hardware processor, when the intersection angle exceeds the threshold angle,
performs processing of a mode selected by the user from a first mode of setting the threshold angle as the angle correction value and providing notification of the setting, a second mode of changing the beam direction of the second ultrasonic beam so that the intersection angle may be equal to or smaller than the threshold angle, and a third mode of informing the user of an operation content of setting the intersection angle to or below the threshold angle.

19. A control method for an ultrasonic diagnostic apparatus, the method comprising:
generating a tomographic image showing an inside of a subject, based on a received signal relating to an ultrasonic echo of a first ultrasonic beam transmitted to the subject;
detecting a Doppler shift in frequency from a transmission frequency of a second ultrasonic beam, based on a received signal relating to an ultrasonic echo of the second ultrasonic beam transmitted to the subject;
detecting a blood vessel position in the subject based on image information about the tomographic image and setting an angle correction value relating to an intersection angle between a blood vessel extending direction at the blood vessel position and a beam direction of the second ultrasonic beam; and
generating a Doppler spectrum image representing a distribution of blood flow rate at the blood vessel position, based on the Doppler shift in frequency and the angle correction value,
wherein when the intersection angle exceeds a threshold angle, the threshold angle is set as the angle correction value and notification of the setting thereof is provided.

20. A non-transitory recording medium storing a computer readable control program for causing an ultrasonic diagnostic apparatus to execute processing, comprising:
generating a tomographic image showing an inside of a subject, based on a received signal relating to an ultrasonic echo of a first ultrasonic beam transmitted to the subject;
detecting a Doppler shift in frequency from a transmission frequency of a second ultrasonic beam, based on a received signal relating to an ultrasonic echo of the second ultrasonic beam transmitted to the subject;
detecting a blood vessel position in the subject based on image information about the tomographic image and setting an angle correction value relating to an intersection angle between a blood vessel extending direction at the blood vessel position and a beam direction of the second ultrasonic beam; and
generating a Doppler spectrum image representing a distribution of blood flow rate at the blood vessel position, based on the Doppler shift in frequency and the angle correction value,
wherein when the intersection angle exceeds a threshold angle, the threshold angle is set as the angle correction value and notification of the setting thereof is provided.
